(19) United States
(12) Patent Application Publication
(10) Pub. No.: US 2009/0240046 A1
(43) Pub. Date: Sep. 24, 2009

(54) CARBOXYLATED GLUCOSAMINE COMPOUNDS FOR INHIBITING THE ACTIVATION AND EXPRESSION OF MATRIX METALLOPROTEINASE-9 IN HUMAN FIBROSARCOMA CELLS AND MATRIX METALLOPROTEINASE-9 INHIBITORS CONTAINING THE SAME

(76) Inventors: Moon-Moo Kim, Busan (KR); Se-Kwon Kim, Busan (KR); Eresha Mendis, Busan (KR)

Correspondence Address: BIRCH STEWART KOLASCH & BIRCH, PO BOX 747, FALLS CHURCH, VA, 22040-0747, US

(21) Appl. No.: 11/908,595

(22) PCT Filed: Sep. 14, 2006
(86) PCT No.: PCT/KR06/03670

§ 371 Date: Sep. 13, 2007

(30) Foreign Application Priority Data
Jun. 22, 2006 (KR) 10-2006-0056556

(51) Int. Cl. C07H 5/06

(52) U.S. Cl. 536/55.2

(57) ABSTRACT

The present invention relates to the CGlc compound for inhibiting the expression and activation of MMP-9 in HT1080 and the MMP-9 inhibitor comprising the CGlc compound as an effective ingredient, which can inhibits the expression of AP-1 in Ht1080 to effectively inhibit the expression and activation of MMP-9 such that it is effectuated in treating a wound, metastasis, rheumatoid arthritis, inflammation, hyperparathyroidism, diabetes, cornel ulcer, osteoporosis, stomach ulcer, trauma, pimple, burn, periodontal disease, arteriosclerosis or fracture.

### TECHNICAL FIELD

The present invention relates to a CGlc compound as MMP-9 inhibitors, more specifically to a CGlc compound of formula 1 for inhibiting the activation and expression MMP-9 in HT1080 and a MMP-9 inhibitor containing the CGlc compound as an effective constituent.

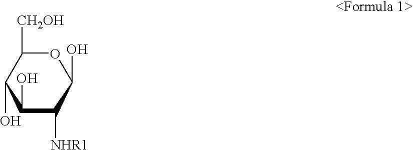

wherein,

R1 is COCH₂CH₂COO—.

Abbreviations which are generally used in the present invention, have the following meanings and without particular definition, it has a meaning of commonly used in the art.

MMP: matrix metalloproteinase

CGlc: Carboxylated glucosamine

HT1080: human fibrosacroma cells

PMA: phorbol 12-myristate 13-acetate

FBS: fetal bovine serum

DMEM: Dulbecco's modified Eagle's medium

MTT: 3-(4,5-dimethyl-2-yl)-2,5-diphenyltetrazilum bromide

AP-1: Activator protein-1

### BACKGROUND ART

The proteolytic degradation of extracellular matrix proteins has long been described in association with both normal tissue remodeling and pathologic conditions. The matrix metalloproteinases (MMPs), which represents a family of major matrix degrading enzymes play a central role in the breakdown of these structural proteins. MMPs share certain biochemical properties, yet each has distinct substrate specificity and up to date several mammalian enzymes have been identified ranging from well-characterized enzymes such as collagenase, stremolysin, gelatinase and more recently described membrane type MMPs. Under normal physiological processes MMP expression and activity is carefully regulated and loss of control of MMP activity appears to have serious consequences and aberrations in MMP expression have been associated with several diseases.

In this context, MMP inhibitors have been caught the interest as an important new class of therapeutic agents for the treatment of diseases characterized by remodeling and excessive extracellular matrix degradation.

Among MMPs, MMP-9 (gelatinase B, 92 kDa) is an important member of the MMP family and is involved in the cleavage of all types of denatured collagens and of native basement membrane proteins. Studies have shown that MMP-9 levels are significantly elevated during the metastatic progression of malignant tumors since it requires proteolytic degradation of ECM components in basement membrane and stroma tissues. HT1080 cells, a human fibrosarcoma cell line have been used extensively as a model to study MMP-9 activity and expression. Similar to various other malignant tumors these cells express MMP-9 at a higher level. Studies on the promoter of MMP-9 have clearly identified that its transcription is mediated mainly via AP-1 transcription factor binding interactions. Compounds that interfere the up-regulation of MMP-9 expression can remarkably decrease the total MMP-9 activity and identification of MMP-9 inhibitors is of current interest.

The earliest MMP inhibitors were peptide derivatives designed from the knowledge of the amino acid sequence of collagen at the site of cleavage by collagenase. With the development in the field, medicinal chemists. focused away from the peptidic inhibitors because of the difficulties in obtaining good oral activity and lack of specificity for MMPs.

As a result, a number of non-peptidic inhibitors have been discovered by high-throughput screening of natural product and also potent MMP inhibitors have been synthesized with desirable chemical functional groups.

Glucosamine is an amino monosaccharide found in chitin, glycoproteins and glycosaminoglycans such as hyaluronic acid and heparan sulfate. Most of the clinical studies investigating medical effects of glucosamine have used glucosamine sulfate and a great trend exists to use it for treating osteoarthritis.

Recently results of some studies suggested that, N-acetyl glucosamine has shown promise for treating inflammatory bowel diseases such as Crohn disease and ulcerative colitis and also glucosamine has been proposed to be important for healthy skin. However, reports on synthesis of other derivatives of glucosamine and identification of their plausible biological activities cannot be found so often.

The present inventors established the fact that a compound carboxylated with glucosamine hydrochloride can inhibit the activation and expression MMP-9 in human fibrosacroma cells, and by examining it to result to the present invention.

### DISCLOSURE

### Technical Problem

Accordingly, the object of the present invention can be achieved in that a carboxylated glucosamine compound is synthesizing by carboxylating glucosamine hydrochloride with succinic anhydride to provide MMP-9 inhibitor capable of inhibiting the expression and activation of MMP-9 in HT1080.

### Technical Solution

The present invention provides a CGlc compound of formula 1 capable of inhibiting the the expression and activation of MMP-9 in HT1080.

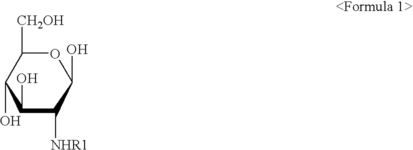

wherein,

R1 is COCH₂CH₂COO—.

The present invention further provides a MMP-9 inhibitor comprising the CGlc compound of formula I as effective ingredient. The MMP-9 inhibitor can be prepared in the form of spray, tablet, capsule, powder, ointment composition, solution, gel, paste, patch or granule.

The present invention further provides a medicinal use of the MMP-9 inhibitor. Diseases, for example a wound, metastasis, rheumatoid arthritis, inflammation, hyperparathyroidism, diabetes, cornel ulcer, osteoporosis, stomach ulcer, trauma, pimple, burn, periodontal disease, arteriosclerosis or fracture can be treated and prevented by the MMP-9 inhibitor of the present invention

As shown by the scheme 1, the CGlC compound of the present invention can be obtained by chemically reforming glucosamine hydrochloride, in which the potential of reverse effect in the structure change is excluded since the reaction is carried out under the mild condition.

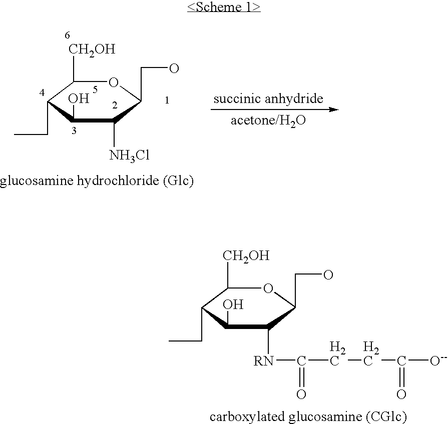

Accordingly, the replacement of COCH₂CH₂COO— is mainly carried out under the controlled condition.

In the followings, the construction of the present invention is described more specifically by the example. However, the scope of the present invention cannot be districted by the example.

### EFFECT OF THE INVENTION

The carboxylated glucosamine compound of the present invention can inhibits the expression of AP-1 in HT1080 to effectively inhibit the expression and activation of MMP-9 such that it is effectuated in treating a wound, metastasis, rheumatoid arthritis, inflammation, hyperparathyroidism, diabetes, cornel ulcer, osteoporosis, stomach ulcer, trauma, pimple, burn, periodontal disease, arteriosclerosis or fracture.

### BRIEF DESCRIPTION OF THE FIGURES

FIG. 1 is graph showing FT-IR spectra of (A) Glc and (B) CGlc.

FIG. 2 is graph showing effect of CGlc on the gelatinolytic activity of MMP-9 in HT1080 cell line.

FIG. 3 is graph showing cytotoxic effect of CGIc (A) in the presence of FBS (B) in the absence of FBS.

FIG. 4 is graph showing detection MMP-9 activity in CGlc treated HT 1080 cells by fluorescence conjugated gelatin digestion inhibition.

FIG. 5 is graph showing effects of CGIc on (A) promoter activity of MMP-9 (B) AP-1 transcriptional activity of HT1080 cells transfected with MMP-9 promoter containing pGL3 luciferase reporter vector and AP-1 binding site containing luciferase reporter vector respectively.

### BEST MODE FOR CARRYING OUT THE INVENTION

### Example 1

Chemicals required for synthesis, including succinic anhydride were obtained from Sigma Chemical Co. (St. Louis, Mo., USA). HT1080 cells were obtained from American Type of Culture Collection (Manassas, Va., USA). All the materials required for culturing of cells including. cell culture media were purchased from Gibco BRL, Life Technologies (USA).

FITC-Gelatin (CLN-100) was obtained from Collagen Technology Corporation (Tokyo, Japan). MTT reagent, gelatin, agarose, doxycycline, PMA (phorbol 12-myristate 13-acetate) were purchased from Sigma Chemical Co. (St. Louis, Mo., USA).

The N-carboxybutyrylation reaction was carried out according to Ronghua et al. with slight modifications. Briefly, Glucosamine hydrochloride (2 g) was dissolved in 10 mL of distilled water and 15 mL of methanol was added while stirring. A determined quantity of succinic anhydride to obtain the same molar ratio (1 g) was dissolved in acetone and added drop by drop at room temperature for 1 h. The mixture was stirred for 4 h and pH was adjusted to 9.0-10.0 with sodium carbonate while reaction. Subsequently, the solution was purified and lyophilized to obtain fluffy, yellow, light solid products.

Proton NMR (¹H NMR) and carbon NMR (13C NMR) spectra were recorded in a D₂O environment on a JNM-ECP-400 (400 MHz) spectrometer (JEOL, Japan). Elemental analysis (C, N and H) was performed using an elementar analysesysteme, (Elementar Vario, EL, USA) and were within ±0.4% of theoretical values. Infrared spectra were recorded using KBr plates in Spectrum 2000 FT-IR spectrophotometer (Perkin Elmer, USA).

Human fibrosarcoma cells (HT1080) were cultured in DMEM supplemented with 10% fetal bovine serum, 2 mM glutamine and 100 μg/mL penicillin-streptomycin. For experiments, cells were detached with trypsin-EDTA and plated onto 24- or 96-well plates at a plating density of 7×10⁵ and 1.5×10⁵ per well, separately.

Equal number of HT1080 cells (4000 cells per well) cultured in 96-well plates with serum and serum free media were treated with various concentrations of CGlc for 24 h. Cell viability was then evaluated with the 3-(4,5-dimethyl-2-yl)-2,5-diphenyltetrazolium bromide (MTT) spectrophotometrically (GENios® microplate reader, Tecan Austria GmbH, Austria) which measures conversion of tetrazolium salt in to blue formazan by viable cells.

MMP-9 activity in HT1080 cells was assayed by gelatin zymography as described previously. For this, approximately 2×10⁵ cells/mL HT1080 cells in serum free DMEM medium were seeded in 24-well plates and pre-treated with different concentrations of CGlc for 1 h. MMP-9 expression was stimulated by treatment of PMA (10 ng/mL) and cells were cultured for 48 h. Cell conditioned medium was subjected to substrate gel electrophoresis. Similar amount of protein containing conditioned media were applied without reduction to a 10% (w/v) polyacrylamide gel impregnated with 1 mg/mL gelatin. After electrophoresis, gel was washed in 50 mM Tris-HCl (pH 7.5) containing 2.5% Triton X-100 at room temperature and incubated overnight at 37° C. in zymography developing buffer containing 10 mM CaCI₂, 50 mM Tris-HCl, and 150 mM NaCl. The gel was then stained with a solution of 0.1%. In this assay clear zones against the blue background indicate the presence of gelatinolytic activity.

The image of the gel was recorded using Fujifilm Image Reader LAS-3000 software in Science Image System, LAS 3000 (Fujifilm Life Science, Tokyo, Japan). The gelatinolytic activities were quantified using Multi Gauge V3.0 software.

HT1080 cells grown in 24-well plates were treated with different concentrations of CGlc and conditioned media were collected by centrifugation at 13 000×g following PMA (10 ng/mL) treatment. Enzyme activities of conditioned media in 50 mmol/l Tris-HCl buffer (pH 7.5), 0.15 mol/l NaCl, 10 mmol/l CaCl₂, 0.02% NaN₃ (TNC buffer) containing 0.05% Brij 35 were determined using fluorescence conjugated gelatin peptide (Collagen Technology Corporation, Tokyo, Japan) as the substrate.

Fluoropeptides were incubated with conditioned media at 37° C. for 20 h, and the reaction was terminated by addition of 3% acetic acid. Fluorescence intensity was measured at 495 nm (excitation) and 520 nm (emission) with a GENios® fluorescence microplate reader (Tecan Austria GmbH, Austria).

HT1080 cells cultured in 10 cm culture dishes were transiently transfected with MMP-9 promoter containing pGL3 luciferase reporter vector (Promega, Madison, Wis.) or AP-1 binding site containing luciferase reporter plasmid (Colontech, Palo Alto, Canada) by Lipofectamine™ 2000™ reagent (Invitrogen). Beta-galactosidase expression vector co-transfected with the reporter vector to serve as an internal control of transfection efficiency. Transfected cells sub-cultured in 24-well plates were treated with different concentrations of CGlc. Following stimulation with PMA cells were cultured for 24 h. Cells were washed once with cold PBS and lysed with 200 μl/well lysis buffer (25 mM Tri-HCl, pH 8.0, containing 2 mM DDT and 1% Triton-X 100). Aliquots of cell lysate and luciferase substrate (Promega) were mixed in equal amounts in a 96-well plate and luminescence intensity was measured with a luminescence microplate reader (Tecan Austria GmbH, Austria). Beta-galactosidase activity was measured with ONPG buffer. The level of reporter gene expression was determined as a ratio, compared with cells stimulated by PMA (10 ng/mL) alone and represented as relative luciferase activity.

Transfection efficiency was determined by X-Gal staining method. Briefly, transfected cells were fixed with 0.5% glutaraldehyde and stained with X-Gal solution containing 20 mM K₃Fe(CN)₆, K4Fe(CN)₆ and 1 mM MgCl₂. After 24 h of incubation at 37° C., transfected cells were visualized with blue color under a light microscope.

Data were expressed as mean ± standard error of the mean (n=3). Student's t-test was used to determine the level of significance at P<0.05.

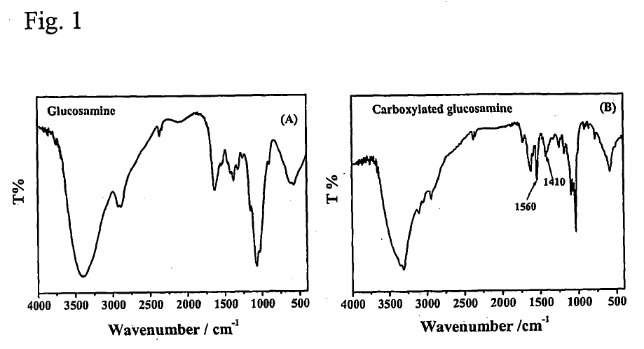

In the present invention, glucosamine was chemically modified to obtain CGlc (Scheme 1) and the reaction was carried out under mild conditions to rule out possibilities for adverse influences on structural changes. Therefore, the substitution of —OOC— CH2CH2—CON— was predominantly under control. Substitution of —OOC—CH2CH2—CO— group to glucosamine was clearly confirmed by FT-IR spectra, Elemental analysis, ¹³C NMR and ¹H NMR of CGlc. In comparison to the FT-IR spectrum of Glc (FIG. 1A), both symmetric and asymmetric stretch absorptions of carboxyl groups (1560 cm-1 and 1410 cm-1 respectively) in spectrum of CGlc (FIG. 1B) confirmed the successful introduction of the new group. Data obtained from elemental analysis greatly strengthened the new structural features observed in IR spectra and in agreement with the calculated elemental composition.

TABLE 1

| | Carbon | | Hydrogen | | Nitrogen | |
|---|---|---|---|---|---|---|
| | content (%) | | content (%) | | content (%) | |
| | Anal. | Cal. | Anal. | Cal. | Anal. | Cal. |
| Glc | 33.48 | 33.42 | 6.61 | 6.54 | 6.49 | 5.51 |
| CGlc | 39.79 | 39.87 | 5.40 | 5.35 | 4.71 | 4.65 |

Information of ¹³C NMR spectra of the derivative was also in agreement with FT-IR spectra and clearly confirmed the substitution of carboxyl groups to CGIc. In ¹³C NMR spectra of Glc, characteristic peaks (δ93 ppm, C-1; δ54 ppm, C-2; δ69 ppm, C-3, δ72 ppm, C-4; δ76 ppm, C-5, δ60 ppm, C-6) were clearly observed. Compared to Glc, CGlc presented some new chemical shifts that can be assigned to COCH₂CH₂COO— group at δ180 ppm, δ177 ppm, and δ32 ppm (assigned to the carbonyl carbon —COO, —CO, and —CH₂CH₂—, respectively). Further, ¹H NMR spectrum of CGlc was used to confirm the existence of substituted groups compared to 1H NMR spectrum of Glc (2.9, 3.2, 3.8, and 4.5 (C-1-6)). A new chemical shift that appeared in spectrum of CGlc-3 at δ2.5 ppm was assigned to protons of COCH₂CH₂CO— groups.

To analyze the inhibition of MMP-9 mediated gelatinolytic activity in conditioned medium stimulated with PMA, we utilized human fibrosarcoma cell line, HT1080. Cells were treated with various concentrations of CGlc and following stimulation with PMA incubated for 3 days. Gelatin zymography was performed as described under methods.

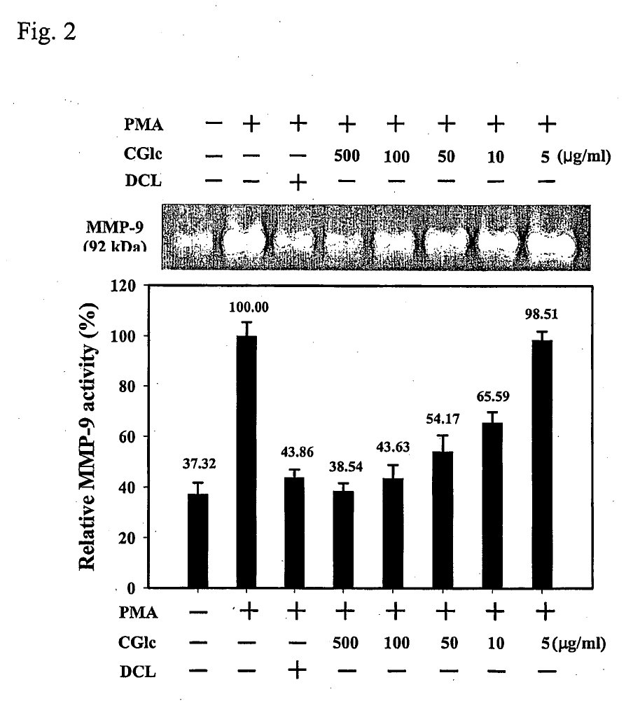

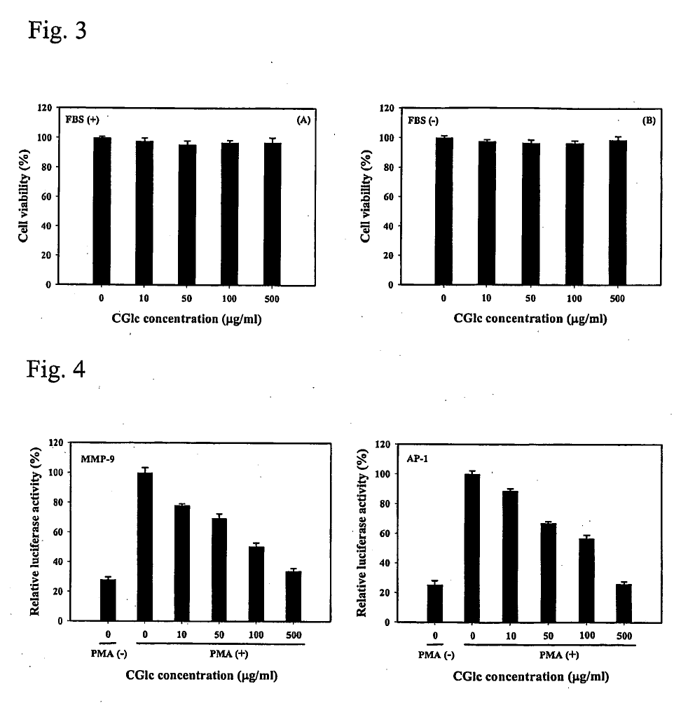

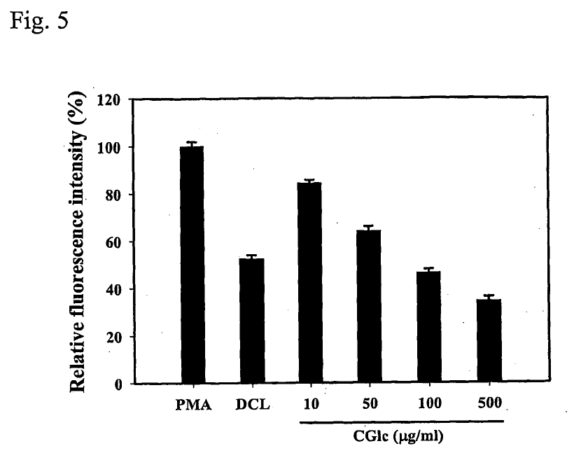

As shown in FIG. 2, similar to various other malignant tumors, these HT1080 cells express MMP-9 at a higher level. When PMA was administered to HT1080 cells at 10 ng/mL concentration MMP-9 activity increased by approximately 60%. Size and the intensity of the lytic zone was greatly reduced in the presence of CGlc and it clearly indicated that MMP-9 activity in HT1080 cells was markedly inhibited in the presence of CGlc. Moreover, inhibitory effect of CGlc on MMP-9 showed a concentration dependant pattern and at concentration 500 μg/mL, CGlc showed approximately 60% inhibition of MMP-9 lead gelatinolytic activity which was much clear than the effect of doxycycline (at 10 μg/mL concentration), a tetracycline analogue we used in this study as the positive control to compare the inhibitory effects. CGlc did not show any toxicity on HT1080 cells even at 1 mg/mL concentration (FIG. 3A, 3B). Therefore, the viability data clearly confirmed that cytotoxicity did not contribute to the observed MMP-9 inhibitory potential of CGIc.

The MMP-9 inhibitory effect of CGlC on MMP-9 activity stimulated by PMA in HT1080 cells was further confirmed using more sensitive fluorescence conjugated gelatin digestion assay which use fluorescence conjugated gelatin peptides as the substrate for MMP-9. The amount of active gelatinases (MMP-9/-2) present in CGIc treated stimulated conditioned medium obtained from incubation of HT1080 cells was assesssed from the resulting fluorescence activity. As we observed clearly in gelatin zymography stimulated with PMA, HT1080 cells secrete mainly MMP-9 and expression of other gelatinases including MMP-2 are negligible. Therefore it can be presumed that difference in fluorescence intensities were mainly due to activity of MMP-9. The inhibitory results obtained in the presence of CGlc were in well agreement with zymography results, where dose-dependent MMP-9 inhibition was observed with increment of concentration of CGlc (FIG. 4). At 500 μg/mL concentration of CGlc, relative fluorescence activity decreased up to 66% and when CGlc was treated at 100 μg/mL concentration about 50% reduction of fluorescence intensity was observed.

To extend these observations to interactions within the context of transcriptional regulation, we assessed the effect of CGIc on expression of MMP-9 using MMP-9 promoter luciferase construct. A high (about 4-fold) luciferase activity was noted in control cells stimulated with PMA and it was well correlated with zymography. However, the activity of the MMP-9-luciferase reporter gene was inhibited nearly 3-fold in the cells treated with CGIc at 500 μg/mL concentration (FIG. 4A). Approximately 50% inhibition was resulted from 100 μg/mL concentration of CGlc and inhibition followed a dose-dependant pattern. We were curious to know whether the MMP-9 transcription suppression effect of CGlc had any correlation to AP-1 transcription. Because the promoter region of the MMP-9 contains an AP-1 binding consensus site upstream from the start site and transcriptional induction of MMP-9 has been demonstrated to be essentially mediated via binding of AP-1 transcription factor to promoter. Therefore, to investigate whether CGIc exert its effect by interfering AP-1 transcription factor binding we transfected cells with AP-1 binding site containing luciferase constructs. Reporter activity in PMA stimulated group was increased by approximately 3-fold whereas reporter activity was suppressed between 2-3 fold once the cells were treated with CGlc at 500 μg/mL concentration. And the suppression followed a clear dose-dependant pattern. Taken together; these experiments suggest that CGIc suppresses MMP-9 expression via suppression of AP-1 expression.

The present is the first to demonstrate the activity of glucosamine derivative that can inhibit the MMP-9 activity and expression. Many of synthetic and natural MMP inhibitors identified so far have proven their capability to direct inhibition of MMP enzymatic activities due to presence of desirable functional groups. Compounds that can exert an effect at transcriptional level could be expected to have a promising potential to develop potent inhibitors since that can interfere the up-regulation of MMP-9 expression and thereby can remarkably decrease the total MMP activity. Therefore CGIc represents a potential synthetic MMP-9 expression inhibitor that warrants further investigation.

### INDUSTRIAL AVAILABILITY

The carboxylated glucosamine compound of the present invention can inhibit the expression of AP-1 in HT1080 to effectively inhibit the expression and activation of MMP-9 such that it is effectuated in treating a wound, metastasis, rheumatoid arthritis, inflammation, hyperparathyroidism, diabetes, cornel ulcer, osteoporosis, stomach ulcer, trauma, pimple, burn, periodontal disease, arteriosclerosis or fracture. Accordingly, the present invention is a medicalpharmaceutically useful invention.

1. A carboxylated glucosamine (CGlc) compound of formula 1 for inhibiting the expression and activation of matrix metalloproteinase-9 (MMP-9) in human fibrosacroma cells (HT1080). 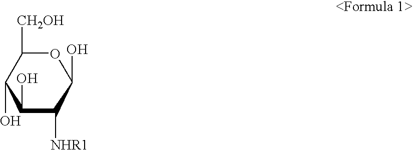
wherein,
R1 is COCH₂CH₂COO—.

2. A MMP-9 inhibitor comprising the CGIc compound of claim 1 as an effective ingredient.

3. The MMP-9 inhibitor according to claim 2 characterized in producing in the form of spray, tablet, capsule, powder, ointment composition, solution, gel, paste, patch or granule.

4. The MMP-9 inhibitor according to claim 2 for the treatment of a wound, metastasis, rheumatoid arthritis, inflammation, hyperparathyroidism, diabetes, cornel ulcer, osteoporosis, stomach ulcer, trauma, pimple, burn, periodontal disease, arteriosclerosis or fracture.
